(19) United States
(12) Patent Application Publication
(10) Pub. No.: US 2005/0273140 A1
(43) Pub. Date: Dec. 8, 2005

(54) Lumbar traction device

(75) Inventors: Ara Boghosian, Alexandria, VA (US); Christian Hunt, McLean, VA (US); John A. Spadavecchia, Red House, VA (US)

Correspondence Address: GREENBLUM & BERNSTEIN, P.L.C., 1950 ROLAND CLARKE PLACE, RESTON, VA, 20191, US

(73) Assignee: Care Rehab & Orthopedic Products, Inc., McLean, VA (US)

(21) Appl. No.: 11/203,992

(22) Filed: Aug. 16, 2005

Related U.S. Application Data
(63) Continuation of application No. 29/193,442, filed on Nov. 12, 2003.

(51) Int. Cl. A61F 5/00

(52) U.S. Cl. 606/241; 602/32

(57) ABSTRACT

A lumbar traction device comprises a first frame member having a first handle portion and a second frame member attached to the first frame member by a hinged member. The second frame member has a second handle portion. The first frame member and the second frame member are moveable between an open position and a closed position. The first handle portion and the second handle portion are substantially aligned to form a first handle when the first frame member and the second frame member are in the closed position.

### CROSS REFERENCE TO RELATED APPLICATIONS

This application is a continuation of application Ser. No. 29/193,442, filed on Nov. 12, 2003, which is now incorporated by reference in its entirety.

### FIELD OF THE INVENTION

The invention generally relates to a lumbar traction device and more particularly to a portable lumbar traction device.

### BACKGROUND OF THE INVENTION

Traction devices are used to relieve pressure on inflamed or enlarged nerves. Cervical and lumbar or spinal traction are the most common type of devices. When correctly performed, the traction devices can relieve pain in the neck and the spine by, for example, straightening the curvature of the spine or stretching of the spinal and cervical musculature.

Portable traction devices are now becoming very popular for in home traction use. This allows patients to perform traction therapy without leaving their homes, or expending large sums of money for a healthcare provider or physical therapist.

Known portable traction devices include pneumatic cylinders controllable by a hand pump. However, known portable lumbar traction device, currently manufactured and sold, are cumbersome and difficult to transport. These lumbar traction devices are basically a large board of approximately 3 or more feet in length.

In one known portable lumbar traction device, two flat separate boards are assembled in order to form the platform for the device. To make such assembly, hooks extending from one flat board are aligned with holes in the other flat board. This is accomplished, most typically, by lifting and aligning the board with the hooks and then inserting the hooks into the holes. During this assembly, a piston rod of a pneumatic device remains attached to one board and the cylinder housing of the pneumatic device remains attached to the other board, making for a very awkward assembly. To disassemble the traction device, the hooks must be removed from the holes, which is an awkward process, especially in view of the piston rods and cylinders remaining attached between the separated boards.

In this lumbar traction device, a rail or track system is screwed or otherwise fastened to one of the flat boards. A carriage is seated within the piston rods of the pneumatic cylinders mounted to the moveable carriage in order to provide the traction force, via a hand pump. All other components are also fastened, in some manner, to the boards. For example, nylon handles are attached to the board so that when the traction device is collapsed, it can be transported by the user.

### BRIEF SUMMARY OF THE INVENTION

In a first aspect of the invention, a lumbar traction device comprises a first frame member having integrally formed sidewalls forming a cavity. A carriage member is seated within the cavity. A second frame member is hinge mounted to the first frame member by a fixed hinged member such that the first frame member and the second frame member are moveable between an open position and a closed position. In aspects of the invention, a first handle portion and a second handle portion are integrally formed with the frame.

In a second aspect of the invention, a lumbar traction device includes a clamshell-like frame extendable to an open position and collapsible to a closed position. The clamshell-like frame includes a first frame member fixedly hinge mounted to a second frame member. In the closed position, the first frame member and the second frame member face one another and, in the open position, the first frame member and the second frame member extend along a longitudinal axis in a single plane. A first and second handle portion are integrally formed with the first frame member and the second frame member, respectively, such that, when the clamshell-like frame is in the closed position, the first and second handle portion form a single handle.

In a third aspect of the invention, a lumbar traction device includes a frame extendable to an open position and collapsible to a closed position, the frame includes a first frame member fixedly hinge mounted to a second frame member. In the open state, the first frame member and the second frame member extend along a longitudinal axis in a single plane. A moveable carriage is seated within a cavity integrally formed in the first frame member. At least one spring is positioned under the moveable carriage.

### BRIEF DESCRIPTION OF THE DRAWINGS

FIG. 1 shows a top view of the lumbar traction device in an expanded position in accordance of the invention;

FIG. 2 shows a bottom view of another embodiment of the lumbar traction device in an expanded position in accordance of the invention;

FIG. 3 shows a perspective view of the lumbar traction device in a closed or collapsed position in accordance with the embodiment of FIG. 2, and

FIG. 4 shows an end view of the lumbar traction device in the closed or collapsed position.

### DETAILED DESCRIPTION OF EMBODIMENTS OF THE INVENTION

The invention is directed to a lumbar traction device. The lumbar traction device is collapsible and includes adjustable features as discussed below. In one implementation, many features of the lumbar traction device are integrally molded with the frame members. The lumbar traction device is portable by collapsing the lumbar traction device into a clamshell-like structure. In the collapsed position, the frame members face one another; whereas, in the open state, the frame members extend along a longitudinal axis in a single plane. The lumbar traction device can also be easily transported by integrally formed handles, for example, on the sides or end thereof.

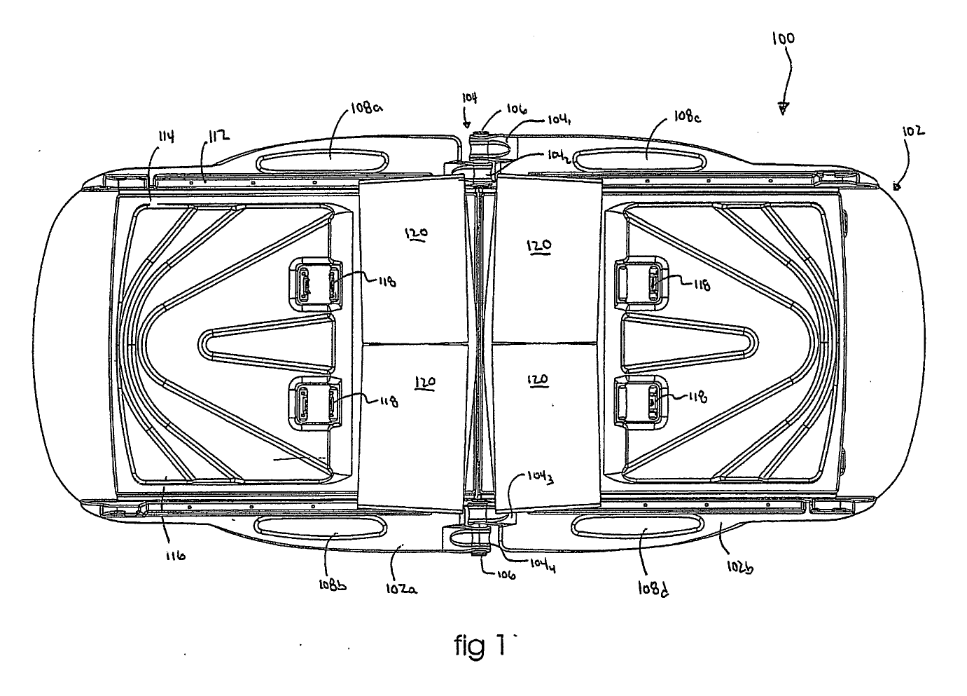

Referring to FIG. 1, a schematic diagram of the lumbar traction device is shown. The lumbar traction device is generally depicted as reference numeral 100 and includes a separate integrally formed frame member 102. The frame member 102 may be a molded plastic, or other known materials used for the construction of lumbar traction devices. The frame 102 is, in embodiments, two separate integrally molded members, depicted as reference numerals 102a and 102b. The frame members 102a and 102b are designed to house or mount thereon many of the components of the lumbar traction device such as a pneumatic type device (not shown).

The frame members 102a and 102b are hinge mounted to one another at hinges generally shown as reference numeral 104. In other words, the lumbar traction device of the invention includes a fixed hinge device, which enables the use to fold the lumbar traction device, without the need to separate the frame members at any time. Also, the design of the frame members allows for the easy folding, e.g., collapsing, and opening of the lumbar traction device without the requirement for aligning any parts, lifting any of the parts or separating any of the parts. In the latter instance, the fixed hinge construction allows the frame members to fold and unfold without disassembly of any of the parts.

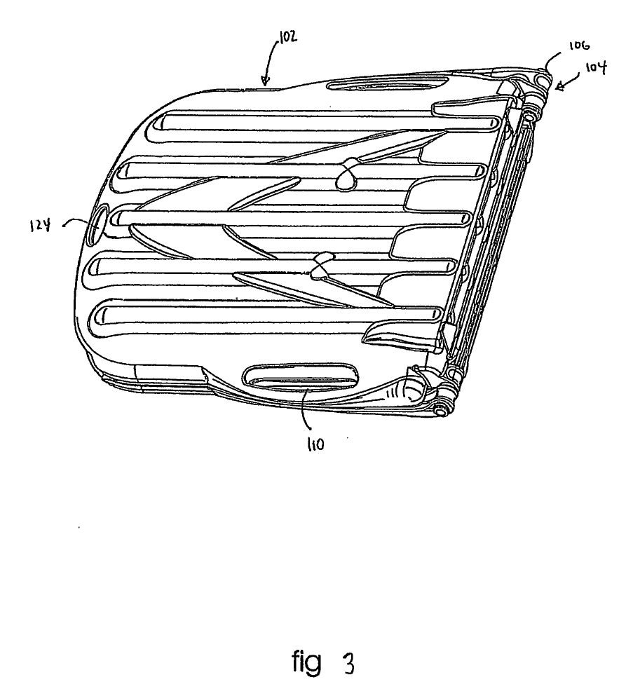

In the open state, the frame member 102a and 102b extend in a single plane. The hinges 104 may be molded onto the separate frame members 102a and 102b such that, for example, the hinges are alternating in position for each of the separate frame members 102a and 104b. By way of one example, from the top of FIG. 1, the hinges may be arranged as hinge member 104₁ of the frame member 102b, hinge member 104₂ of frame member 102a, hinge member 104₃ of frame member 102b and hinge member 104₄ of frame member 102a. The hinges holds the two frame member together so that one can swing relative to the other, e.g., the hinges 104 allow the lumbar traction device 100 to be opened and closed, as shown in FIG. 1 and FIG. 3, respectively, for example. The hinge members 104₁-104₄ are coupled by a respective hinge pin 106, which remains within the hinge during all aspect of the use.

Still referring to FIG. 1, the lumbar traction device 100, and more particularly, the frame members 102a and 102b include integrally formed handle portions 108a, 108b, 108c and 108d. In the closed or collapsed position, the handle portions 108a and 108b of the frame member 102a align with the handle portions 108c and 108d of the frame member 102b, respectively, thus forming a handle 110 on either side of the lumbar traction device, when in the collapsed position, as shown in FIG. 3.

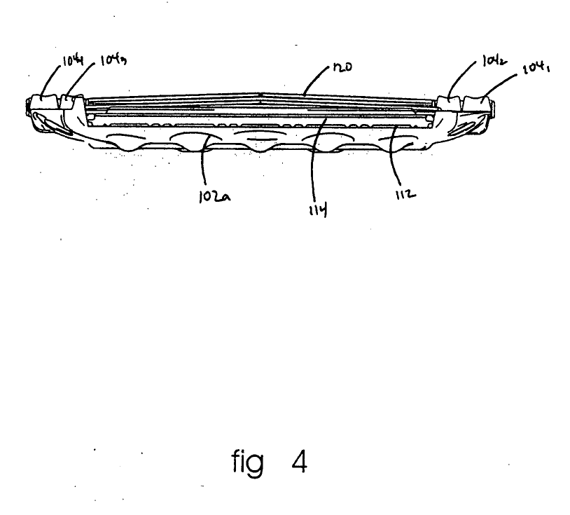

FIG. 1 further shows side walls 112 positioned on opposing sides of the frame member 102a and/or frame member 102b. The side walls 1120 are integrally formed with the frame members 102a and/or 102b and form a cavity 112a in which a moveable carriage 114 is seated therein (See FIG. 4). In one embodiment, the carriage is slidable between the two side walls 112, within the cavity. The cavity 112a includes one open end as depicted at reference numeral 113, formed by the end portions of the integrally formed sidewalls. As one of skill in the art should recognize, the moveable carriage 114 is moveable in directions along the longitudinal axis of the frame 102 in order to provide a traction force. A pneumatic type device (not shown) can be implemented to provide the movements of the moveable carriage 114 and hence provide the traction to a patient or user. A support pad, cushion or other contouring 116 may be mounted onto or attached to the moveable carriage 114.

At least one spring 118 is positioned under the moveable carriage 114. In one embodiment, the at least one spring 118 is two springs. The springs 180 are shown by the cross hatching to better illustrate the position of the springs 118. In one embodiment, the springs 118 are attached to the frame member 102 and the moveable carriage 114, to provide a biasing force. The cushion or other contoured device 116 may be attached to the moveable carriage 114. A pair of belts 122 may be mounted to each of the frame members 102a and 102b.

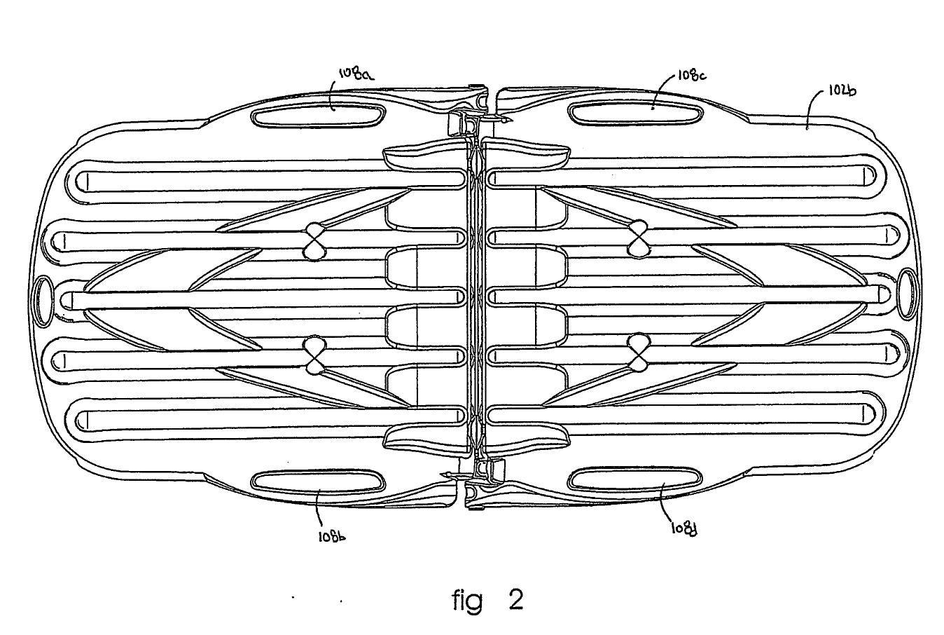

FIG. 2 shows another embodiment of the lumbar traction device of the invention. In FIG. 2, handle portions 122a and 122b are provided at ends of the frame members 102a and 102b. The handle portions 122a and 122b may be integrally molded into the frame members 102a and 102b. In the closed position, as shown in FIG. 3, the handle portions 122a and 122b align with one another, thus forming a handle 124 at an open end, opposing the hinges, as shown in FIG. 3. The handles 110 and 124 facilitate the carrying of the portable lumbar traction device.

FIG. 3 shows the portable lumbar traction device in a closed position. In this position, the handle 124 is shown to be at the open end portion, opposite to the fixed hinged end. The handle 124 is substantially positioned along a longitudinal axis of the lumbar traction device. The handles 110, on the other hand, are shown to be on the sides of the frame members 102a and 102b. This configuration allows a user to hold the portable lumbar traction device either by its side or at the end. Also, in the closed or collapsed position, the portable lumbar traction device is approximately half the original size, thus making it more convenient and easier to transport the portable lumbar traction device. In the collapsed position, the frame members also face one another.

While the invention has been described in terms of preferred embodiments, those skilled in the art will recognize that the invention can be practiced with modification within the spirit and scope of the appended claims.

1. A lumbar traction device, comprising:
a first frame member having integrally formed sidewalls forming a cavity;
a carriage member seated within the cavity, and
a second frame member attached to the first frame member by a fixed hinged member such that the first frame member and the second frame member are moveable between an open position and a closed position.

2. The lumbar traction device of claim 1, wherein:
first frame member includes an integrally formed first handle portion; and
the second frame member includes an integrally formed second handle portion,
the first handle portion and the second handle portion are substantially aligned to form a first handle when the first frame member and the second frame member are in the closed position.

3. The lumbar traction device of claim 2, wherein the integrally formed first handle portion and the second integrally formed handle portion are positioned at a side of the first frame member and the second frame member, respectively, and form the first handle at a side when the first frame member and the second frame member are in the closed position.

4. The lumbar traction device of claim 2, wherein the integrally formed first handle portion and the integrally formed second handle portion are positioned at an end of the first frame member and the second frame member, respectively, and form the first handle at an open end portion thereof.

5. The lumbar traction device of claim 2, further comprising an integrally formed third handle portion on the first frame member and an integrally formed fourth handle portion on the second frame member, the integrally formed third handle portion and the integrally formed fourth handle portion are substantially aligned to form a second handle when the first frame member and the second frame member are in the closed position.

6. The lumbar traction device of claim 5, wherein the second handle is formed at a side or end of the first frame member and the second frame member when in the closed position.

7. The lumbar traction device of claim 1, wherein the fixed hinged member includes alternating hinge portions on the first frame member and the second frame member.

8. The lumbar traction device of claim 7, wherein the alternating hinge portions are four hinge portions, alternating on the first frame member and the second frame member and hinged together by two separate fixed pins.

9. The lumbar traction device of claim 1, wherein the carriage member is moveable within the cavity.

10. The lumbar traction device of claim 9, further comprising a support pad mounted onto the carriage.

11. The lumbar traction device of claim 9, further comprising at least one spring positioned under the carriage.

12. The lumbar traction device of claim 9, further comprising at least one spring attached to the first frame member and the carriage.

13. The lumbar traction device of claim 1, further comprising a belt mounted to at least the first frame member.

14. A lumbar traction device, comprising:
a clamshell-like frame extendable to an open position and collapsible to a closed position, the clamshell-like frame including a first frame member fixedly hinge mounted to a second frame member, in the closed position, the first frame member and the second frame member face one another and, in the open position, the first frame member and the second frame member extend along a longitudinal axis in a single plane; and
a first and second handle portion integral with the first frame member and the second frame member, respectively, such that, when the clamshell-like frame is in the closed position, the first and second handle portion form a single handle.

15. The lumbar traction device of claim 1, wherein the single handle is positioned at least at one of a side of the first frame member and the second frame member and at an end of the first frame member and the second frame member.

16. The lumbar traction device of claim 1, wherein the hinge mount includes four hinged members integrally formed an alternating configuration between the first frame member and the second frame member.

17. The lumbar traction device of claim 14, further comprising a carriage slidably mounted in a cavity of the first frame member and at least one spring positioned under the moveable carriage and attached to the frame and the moveable carriage.

18. A lumbar traction device, comprising.
a frame extendable to an open position and collapsible to a closed position, the frame including a first frame member fixedly hinge mounted to a second frame member, in the open state, the first frame member and the second frame member extend along a longitudinal axis in a single plane;
a carriage is seated, moveably, within a cavity integrally formed in the first frame member; and
at least one spring positioned under the moveable carriage.

19. The lumbar traction device of claim 18, further comprising handle integrally formed in the first frame member and the second frame member and is positioned at least on a side of the frame.

20. The lumbar traction device of claim 18, wherein the first frame member and the second frame member include integrally formed hinges which are hinged together by a fixed, non-removable pin.
